(19) United States
(12) Patent Application Publication
(10) Pub. No.: US 2012/0143018 A1
(43) Pub. Date: Jun. 7, 2012

(54) PORTABLE TOUCHLESS VITAL SIGN ACQUISITION DEVICE

(76) Inventors: Frank M. Skidmore, Gainesville, FL (US); Mark Davidson, Florahome, FL (US); Russell S. Donda, Gainesville, FL (US)

(21) Appl. No.: 13/145,279

(22) PCT Filed: Jan. 19, 2010
(86) PCT No.: PCT/US10/21416

§ 371 Date: Feb. 21, 2012

Related U.S. Application Data
(60) Provisional application No. 61/145,675, filed on Jan. 19, 2009.

(51) Int. Cl. A61B 7/04; A61B 5/01; A61B 5/02; A61B 5/05

(52) U.S. Cl. 600/301; 600/549; 600/586; 600/528

(57) ABSTRACT

Disclosed herein is a non-contact MCG is anticipated as one embodiment. Additionally, a non-contact stethoscope, thermal sensor, or MCG could be utilized singly or in combination with each other, or included singly or together in other medical devices such as a fluoroscope, For example, a handheld, portable instrument comprising a non-contact stethoscope without a magnetometer or thermal sensor can provide a measure of acoustic signals without contacting a subject, while a non-contact thermal sensor as a single device can provide a rapid contactless temperature of a subject

### RELATED APPLICATIONS

This application claims the benefit of U.S. Ser. No. 61/145,67 filed Jan. 19, 2009, under 35 USC §119(e) which is incorporated herein by reference.

### INTRODUCTION

The body produces acoustic, thermal, and electromagnetic signals that can be detected using appropriate instruments. For example, electrocardiography (ECG) has an important and well established role in the diagnosis and management of cardiovascular disease. The ECG provides a high temporal resolution (on the level of milliseconds or better) of signals arising from the human heart. However, the localizing value of ECG is limited. The strength of electrical signals arising from the heart is related to the boundaries and conductivity of underlying tissues, and to the proximity of sources to the electrode contacts. Location of potential maxima on the skin may not overlay signal sources, making it difficult to localize source directly from the electrical potential map. Similarly, cardiac or other sounds, as well as temperature are important clinical measures used to develop basic vital statistics to measure patient well being.

A hand-held device that provides capability for contact-less measurement of vital statistics such as cardiac electromagnetic activity, cardiac and breath sounds, and temperature would be an advance over the current state of the art. Stethoscopes, a common item used in clinic, emergency rooms and in pre-hospital settings to obtain basic vital statistic information, are notorious fomites or carriers of infection. For example, recently it has been estimated that approximately ⅓ of all stethoscopes carried by emergency medial service (EMS) professionals (first responders who interact with accident victims and other seriously ill individuals in the pre-hospital setting) harbor methicillin-resistant Staphylococcus aureus (MRSA).¹ The hospital environment is similarly contaminated; in a recent article out of 110 stethoscopes tested, microbial contamination was present in 92%, with 20% of stethoscopes contaminated with MRSA.² Pathogenic bacteria on stethoscopes have been found in adult inpatient and intensive care unit environments,³ as well as neonatal wards and intensive care units.⁴ MRSA has been shown to survive on dry uncleaned stethoscopes for over 2 weeks.⁵ Other infectious agents, from clostridium difficile⁶ and gram negative rods³, as well as Respiratory Syncytial Virus⁷ and Varicella Zoster (the causitive agent for “Chicken Pox”)⁸ have also been isolated from stethoscopes. Contaminated stethoscopes have been identified in at least one case as the vector for in-hospital outbreaks of serious infection.⁹ Some had suggested that the use of stethoscope diaphragm covers might improve the properties, however a recent study showed that diaphragm covers if anything increased the rate of microbial contamination.¹⁰ Moreover, even regular cleaning protocols did not clear potentially pathogenic bacteria.³ In fact, it is recommended by some authors that stethoscopes “as extensions of the hands should be washed as frequently as the hands”,¹¹ however in other studies it has been demonstrated that health care professionals rarely, if ever, follow recommended decontamination procedures with stethoscopes.^{1,2,3,4,5,6} While surprising, procedures for stethoscope cleaning may add considerable time, and in an environment where efficiency of delivery of health care is important time spent in non-patient care activities is under considerable pressure.

The inventors have realized that a handheld, contactless vital signs monitor would allow health care practitioners to maintain efficiency, and prevent cross-contamination by using a touchless approach to obtain vital signs. Specifically, auditory auscultation of cardiac rhythm and respiratory rates and sounds, as well as temperature, may be obtained without touching the patient. A device incorporating touchless detection of cardiac and breath sounds, respiratory rhythm, or temperature either in one combined device or in separate devices is described.

Obtaining cardiac electromagnetic information may also be incorporated either in a single purpose device or in a combination device capable of assessing cardiac sounds, respiratory rate or sounds, and/or temperature.

Magnetocardiography (MCG) is another tool to measure the electromagnetic signals arising from the heart. MCG also provides a high temporal resolution of signals arising from the heat, however the inventors have realized that the MCG has certain advantages over the ECG as the MCG is developed using sensitive biomagnetometers. Magnetic signals are not distorted or attenuated by passage through overlying tissue. One notable advantage of biomagnetometers realized by the inventors is that physical contact with a tissue is not necessary for a signal to be detected. This enables envisioning a number of applications in MCG, including rapid evaluation, or, for example, continuous monitoring in environments where skin contact is not desirable (e.g. burns) or possible (e.g. in utero fetal heart monitoring). Specialized personnel are not required to place leads, allowing for automated monitoring (for example, as is done with automated blood pressure cuffs in pharmacies). Furthermore, it is other applications are envisioned by the inventors, for example, placement of devices at health clubs or other non-medical settings for screening purposes. MCG can be used to non-invasively explore the fetal heart in utero. Additionally, MCG, unlike ECG, can be used for source localization. For example, multi-channel MCG can be used to noninvasively localize ectopic foci in atrial fibrillation, a procedure that commonly requires invasive testing.

While these advantages would seem to make an MCG ideal as clinical measuring devices, magnetic monitoring has disadvantages that have prevented it from reaching a larger clinical utilization. One significant disadvantage of biomagnetic signals is that signals are relatively weak compared with ambient magnetic disturbances. The magnetic field of the human heart is the strongest biomagnetic generator. With a peak amplitude of about 100 pT, however, fields generated by the heart may still be orders of magnitude smaller than stray environmental fields. Moreover, until recently, the only sensors reliably able to detect magnetic fields in the appropriate range have been superconducting quantum interference device (SQUID) arrays. SQUID-based systems are relatively expensive, requiring cryogenic cooling and bulky, rigid dewars. In the past, magnetic shielding has also been a required significant expense associated with MCG systems. Recently multichannel SQUID arrays have been developed that no longer require shielding owing to the use of gradiometric configurations, suggesting that MCG may be effectively performed without expensive magnetic shielding. However, these devices are still quite expensive in both capital and operational costs. MCG has therefore remained largely a research device, limited to a few academic centers.

The inventors have realized that in order for touchless vital monitoring to develop, substantial alterations to current technology will be necessary. For touchless monitoring of cardiac and breath sounds, development of a new acoustic detector that is able to operate at the scale desired (e.g. a few inches from the chest) with adequate signal amplification and noise rejection. Similarly, in order for MCG to become a commercially viable technology, substantial alterations in the technology would be necessary. For example, the inventors surmise that it would be desirable for the device to operate in an ambient field, to be inexpensive and have low operating cost, and should be easy to use.

In the case of the non-contact stethoscope, the device may consist of an acoustic transducer such as a microphone element placed in some proximity to the subject, but not touching the subject or anything in contact with the subject. The position of the microphone will determine which sounds are detected. Non-contact detection of heart, lung, and other internal sounds is difficult using a simple microphone element. This is due to the small acoustic energy levels transmitted from the body wall to the air, especially in comparison to background noise, for example in a hospital environment. While it is possible to detect internal sounds with just an acoustic transducer, in practice, background noises compete with the signal of interest. There are several ways to improve this situation, which are discussed herein.

In the case of non-contact detection of cardiac electromagnetic fields, recently, optical magnetometer arrays have been developed that are compact, sensitive, and have the necessary dynamic range to both detect the cardiac rhythm and operate in ambient field like the current SQUID based gradiometer devices. Optical Magnetometers may be compact, leading to the possibility of a portable device. Second, optical magnetometers do not require cryogenic cooling, leading to the potential of significant cost savings. Third, optical magnetometers may potentially be easily manufactured on a mass production scale. The inventors propose that an optical magnetometer has the necessary characteristics to allow development of a commercial, low cost, portable MCG. The inventors set forth below how magnetometers may be adapted for use in acquiring medical information, as well as processes and devices that acquire medical information without the need for contacting the patient.

### SUMMARY

The inventors have realized that a handheld, contactless vital signs monitor would allow health care practitioners to maintain efficiency, and prevent cross-contamination by using a touchless approach to obtain vital signs. Specifically, auditory auscultation of cardiac rhythm and respiratory rates and sounds, as well as temperature, may be obtained without touching the patient. A device incorporating touchless detection of cardiac and breath sounds, respiratory rhythm, or temperature either in one combined device or in separate devices is described.

According to one embodiment described herein, the invention pertains to a new type of portable, hand-held device that will allow non-contact sensing of vital signs such as cardiac and breath sounds, temperature, and cardiac electromagnetic impulses either in a combination or single-purpose device. According to another embodiment, the invention pertains to development of a device for contact-less sensing of auditory or electromagnetic information in other settings, including non-medical settings.

In the case of non-contact detection of cardiac or respiratory acoustic sounds, the microphone can be coupled to a sound gathering and/or concentrating device. The concentrating/gathering device serves to increase the level of sound that is detected by the acoustic transducer.

In another embodiment, the combination or single purpose device is in operable communication with a computer and/or smart phone so that patient data is recorded, analyzed, and stored.

One example of a non-contact stethoscope comprises a parabolic concentrator device that serves to receive sound vibrations produced from a larger area of the surface being monitored (such as a chest wall) and an acoustic transducer, such as a microphone. The concentrator device can concentrate those vibrations by focusing the sound waves to the entrance of a microphone. This serves to both provide increased signal at the microphone, and can also add directionality to the stethoscope system, thereby decreasing the background noise signal.

The signal from the sound transducer (which could include electrical, optical, acoustic or other signals) can be sent to an amplifying system. This system will then provide appropriate output signals. This output could be an acoustic signal as the output from a speaker or headphones or earplugs or other acoustical outputs. The output can be sent to a recording device, the output of which can be stored for later use, or further signal processing.

In order to further reduce external noise, the amplifier and/or signal processing can include frequency filtering or signal processing to remove extraneous signals. For example, heart sounds are primarily between 50 and 1000 Hz. Higher frequencies can be rejected with only minimal loss of useful heart sounds. The system can be modified further to provide different filtering/processing to accentuate various signals. For example, one setting might optimize breath sound detection, another gut sounds, and yet another for heart sounds.

In one example of this portion of the non-contact stethoscope, a deep parabolic reflector with a depth of 1-6 inches and an entrance diameter of 1-4 inches, or more specifically about 2.75 inches, is used as a concentration device. A hole in the base of the parabolic shape is placed in such a way that an acoustic transducer (for example, a condenser microphone element) can be placed at the focal point of the parabolic shape.

For further refining of the directional response of the stethoscope, the size of the hole to the acoustic transducer can be modified. This can be accomplished during the manufacture of the parabolic element, or by introduction of various size apertures blocking a portion of the active area of the sound transducer.

In another example of the non-contact stethoscope, a shallow parabolic reflector can be used. In this case, the focal point lies above the plane of the entrance to the parabola or in the parabola at a point not providing convenient mounting of the acoustic receiver at a hole in the base. The acoustic transducer in this case can be mounted on a mounting apparatus in such a way that the sensitive portion of the transducer lies near the focal point of the parabola.

Another method for gathering and focusing the sound is a waveguide. In this case a tapered waveguide can be used to gather and concentrate the acoustic waves.

The concentrator device or other parts of the stethoscope can also be coated with sound blocking materials. These materials can be absorbing or reflecting media. The coating of various elements of the stethoscope with sound blocking material can serve to further reduce extraneous backround signals.

An acoustic waveguide can also be used to receive the acoustic signal from a concentration device (such as a parabolic reflector, for example) and deliver the acoustic signal to a remote location. The remote location could be directly to earpieces, if no further amplification is required, or to an acoustic transducer (such as a microphone element). One advantage of this method is to further decouple the transducer from any reflection, reducing the potential for feedback.

The acoustic transducer is one which translates acoustic energy into a form of energy for further processing. The acoustic transuder may, for example be a conventional electret microphone, condsenser microphone, or other microphone that converts acoustic energy to electrical signals. In addition, for example, the acoustic transuder may be an optical microphone that converts acoustic energy to an optical signal which can be detected and further processed into useful signals.

While the non-contact stethoscope described here is useful for auscultation of patients in a medical setting, the device can be used in many applications where non-contact detection of auditory signals from other subjects are advantageous. One example would be the detection of localized engine sounds. This would be useful for diagnosing and localizing various engine functions. The non-contact would be useful for avoiding contact with moving, hot, or otherwise dangerous or providing access to inconvenient locations.

A non-contact stethoscope or combined device will allow measurement of vital signs without touching a patient, lessening the problem of cross-contamination. A non-contact MCG instrument will allow for the use of an MCG without the complications of large, immobile, expensive, dewars and MCG arrays. A specific embodiment of the contact-less MCG pertains to a stethoscope-like, handheld portable clinical measurement device with adequate sensitivity (often 100 fT/√Hz or less) to detect magnetic signals from a heart, with an appropriate frequency response and dynamic range to potentially operate effectively in an ambient field without needing to contact the subject. In one embodiment, a sensor having a sensitivity of at least 500 fT/√Hz, and frequency response, dynamic range, and spatial response the sensor(s) is measured. As the inventors have a target sensitivity of 500 fT/√Hz, and the peak amplitude of the MCG is on the order of 100 pT/√Hz or greater (during the QRS complex), the inventors believe embodiments possess sufficient sensitivity to detect the MCG. In a more specific embodiment, a device is built for measuring the MCG using a microfabricated sensor crafted according to Schwindt, Kitching, and others who have described sensitive microfabricated “rice grain” chip scale (<12 mm3) atomic magnetometers.^{12,13,14} A sensor could use alternate light sources such as LEDs or other alternate light sources other than lasers.

Another embodiment pertains to a handheld, portable and clinical measurement device comprising a magnetometer for measuring magnetic fields associated with the human heart in a clinical subject, combined with a sensitive non-contact stethoscope to allow simultaneous measurement of magnetic and acoustic signals without needing to touch or contact a subject.

Another embodiment pertains to a handheld, portable and clinical measurement device comprising a magnetometer for measuring magnetic fields associated with the human heart in a clinical subject, combined with a sensitive non-contact stethoscope and a thermal sensor such as an infrared sensor to allow simultaneous measurement of magnetic signals, acoustic signals, and temperature without needing to touch or contact a subject.

A non-contact stethoscope, thermal sensor, or MCG could be utilized singly or in combination with each other, or included singly or together in other medical devices such as a fluoroscope, For example, a handheld, portable instrument comprising a non-contact stethoscope without a magnetometer or thermal sensor can provide a measure of acoustic signals without contacting a subject, while a non-contact thermal sensor as a single device can provide a rapid contactless temperature of a subject,

Safety precautions required for initial clinical testing are anticipated to be minimal, but will include minor electronics changes to allow sensor repositioning, thermal shielding, and possibly a non-magnetic barrier or other safety precautions.

According to another embodiment, a device embodiment includes on its surface or at least a portion thereof a protective anti-microbial coating, such as those known in the art.

According to another embodiment, the invention pertains to an MCG device using a small scale (“meso-scale” (˜2 cm)) atomic magnetometer capable of measuring the cardiac signal. A meso-scale sensor will provide easily detectable signals, but still be small enough for a single sensor to be placed in a hand-held device. A sensor having sensitivity of 500 ft/√Hz or less is utilized. The embodiment pertains to a single-sensor device using optical magnetometers combined with active and passive shielding targeting a sensor sensitivity of 500 fT/√Hz or less. Embodiments of the invention are designed to be lightweight, portable, relatively inexpensive, and will and allow for easy patient repositioning. Dominant sources of system noise may be detected and minimized.

Another example includes the subsequent processing of the MCG signals to provide an output similar to an electrocardiogram output. This provides the operator a more familiar output.

Also, the term “subject” as used herein may pertain to a human or non-human animal. In an alternative embodiment, the term “subject” pertains to a non-animate object including a machine or mechanical device.

The term “stethoscope” unless otherwise specified herein is used in its broadest to pertain to an instrument for obtaining acoustic signals from a subject as defined herein.

### BRIEF DESCRIPTION OF THE DRAWINGS

FIG. 1 shows a side view of a hand-held touchless vital sign acquisition device embodiment.

FIG. 2 shows a top view of the embodiment shown in FIG. 1.

FIG. 3 shows a side view of another hand-held touchless vital sign acquisition device.

FIG. 4 shows a side view of a hand-held touchless vital sign acquisition device having a transducer located in front of a concentration device.

FIG. 5 shows a schematic of a typical magnetometer embodiment that may be adapted for use in conjunction with the teachings herein.

FIG. 6 shows a chip-scale magnetometer that may be adapted for use with embodiments herein: a) Schematic of the magnetic sensor with components 1—VCSEL, 2—optics package including(from bottom to top) a glass spacer, a neutral-density filter, a refractive microlens surrounded by an SU-8 spacer, a quartz l/4 waveplate, and a neutral-density filter, 3—87Rb vapor cell with transparent ITO heaters above and below it, and 4—photodiode assembly. (b) Photograph of a magnetic sensor. [Schwindt et al 2004].

### DETAILED DESCRIPTION

A non-contact MCG is anticipated as one embodiment. Additionally, a non-contact stethoscope, thermal sensor, or MCG could be utilized singly or in combination with each other, or included singly or together in other medical devices such as a fluoroscope, For example, a handheld, portable instrument comprising a non-contact stethoscope without a magnetometer or thermal sensor can provide a measure of acoustic signals without contacting a subject, while a non-contact thermal sensor as a single device can provide a rapid contactless temperature of a subject.

With respect to the non-contact stethoscope, a number of sound pickup techniques could be used for acoustic sensing. There are optically based sound pickup devices using lasers that could use the atomic magnetometer light source to develop the signal. More traditional directionally sensitive sound pickup devices may also be used. The non-contact stethoscope would optimally consist of a sound detection device such as a microphone configured to be highly directional and optionally connected to low-noise, high gain amplifiers to provide audible output from the small sound waves due to heart sounds emanating from the chest. The device is made sensitive enough to provide detectable signals from the vibrations transmitted through the chest wall to the air surrounding the patient so no direct contact is necessary. Optionally, the same device could be used to detect other internal sounds such as lung, gut, or other sounds. The output of the device is not limited to audible output, but could be, for example, be recorded to a visible trace on a paper output, or a computer screen or other output as would be deemed useful to the user such as a clinician or EMS worker for example.

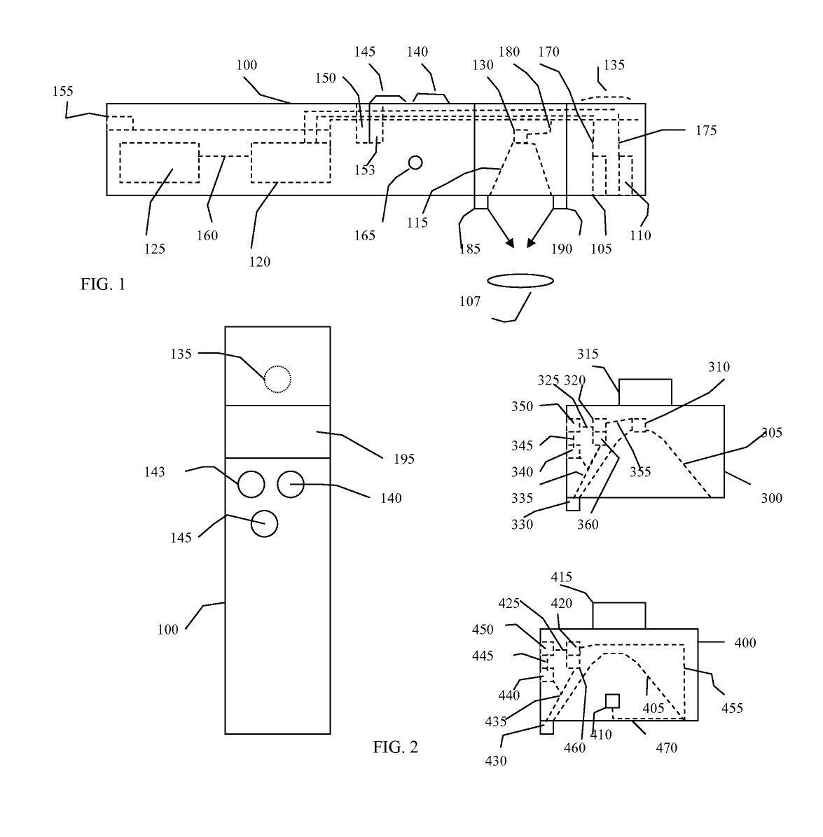

Turning to FIG. 1, a side view of a hand-held portable vital sign acquisition device 100 is shown comprising a concentrator 115, a magnetometer 105 and a thermometer 110. At the inner point of the concentrator 115, a transducer 130 is positioned for transducing sound waves emitted from subject 107 and concentrated by concentrator 115. The magnetometer 105, the thermometer 110 and transducer 130 are communicatingly connected via circuitry 170, 175 and 180, respectively, to processor 120 for processing signal information received therefrom. Signal information may be enhanced by an amplifier component 153 and filter component 150. Signal information either downstream or upstream of processor 120 may be transmitted from the device 100 via connection device 155. The connection device 155 may be a jack for a wire connection such as a unversal serial bus (USB), firewire, ethernet, and the like. In an alternative embodiment, the connection device 155 is a transmitter for sending wirelessly sending information such as, but not limited to, a bluetooth connection device and other conventional wireless connection devices. The device 100 also includes a power source 125 which may take the form of a battery. In a specific embodiment, the battery is rechargeable via connection device 155. The power source 125 is connected to the processor 120 via circuitry 160. Also, included on the device 100 is an earphone jack 165.

Located proximate to the concentrator is 115 are laser components 185 and 190, which may also be connected to processor 120 and/or battery source via circuitry (not shown). The laser components 185 and 190 assist the user in positioning the device 100 at a desired position away from the subject 107. In a specific embodiment, laser beams (arrows) are pointed toward the subject 107 according to a vector wherein the convergence of the beams on the subject 107 indicates an optimized distance for obtaining vital sign information from the subject 107.

In one embodiment, the device 100 is operated by contacting one or more actuators 140 or 145, depending on which function is desired, to initiate the acquisition of vital sign information from the subject 107. For example, if acoustic information is desired, actuator 145 is contacted which initiates acquisition of acoustic information via the transducer 130. Such acoustic information is concentrated by concentrator 115 as it approaches and is impacted on transducer 130. In a specific embodiment, contacting actuator 145 also activates laser components 185 and 190. Acoustic information is processed by processor 120 and may be relayed to user via connection device 155, an ear phone (not shown) via ear phone jack 165 and/or sound emitted by speaker component 135. Acoustic information, or any other signal information generated by device 100 may be recorded by a computer device (not shown), typically as a wired or wireless transmission by way of connection device 155. Actuator 140 is contacted to initiate acquisition of temperature information from the subject 107. In an alternative embodiment, one or both of laser components 185 and 190 are replaced with a sensor involving non-visible light which activates an auditory signal when the device 100 is at the desired proximity to the subject 107.

FIG. 2 shows a top view of the device 100 shown in FIG. 1. The actuators 145 and 140 discussed above are shown, as well as actuator 143 for activating magnetometer 105. The speaker component 135 is also shown. In addition, the device 100 includes a display 195 for displaying information to the user.

FIG. 3 shows a side view of an alternative embodiment 300 of an information acquisition device that has a configuration more like a convention stethoscope. The device 300 includes a handle component 315, a concentrator 305 and a transducer 310. The transducer 310 is connected via circuitry 355 to a processor 320, which is connected to power source 360. Also included on the device 300 is a magnetometer or thermometer component 330 that is also connected to processor 340 and power source 360 via circuitry 335. Processors 320 and 340 are connected to a connection device 350 via circuitry 325 and 345, respectively. The transducer 310 is positioned proximate to an innermost portion of the concentrator 305.

FIG. 4 shows a side view of an alternative embodiment 400 of an information acquisition device that has a configuration more like a convention stethoscope. The device 400 includes a handle component 415, a concentrator 405 and a transducer 410. The transducer 410 is held by a harness component 470 in a frontward position relative to the concentrator 405 and connected via circuitry 455 to a processor 420 which is connected to power source 360. Also included on the device 400 is a magnetometer or thermometer component 430 that is also connected to processor 440 and power source 460 via circuitry 435. Processors 420 and 440 are connected to a connection device 450 via circuitry 425 and 445, respectively. The transducer 410 is positioned relative to the concentrator 305 such that acoustic information is reflected and aimed at the transducer 410.

Those skilled in the art will appreciate that the transducer may positioned at a variety of locations on device relative to the concentrator depending on the type of transducer used. Microphone components are known to have certain angles of reception which will allow the microphone to be placed at a convenient location optimal for receiving acoustic information. Such variety of locations do not necessary relate to those shown in FIGS. 1, 3 and 4.

A variety of methods for detecting thermal signals are also available. Optimally, an infrared sensor in the 5-12 micron wavelength range could be used to detect temperatures in the normal body temperature range. A device, for example, could be directed towards an open orifice such as a mouth to pick up infrared emission from the back of the throat.

Optimizing distance from the signal source may be useful in some embodiments, and therefore in some cases a variety of means may be used to determine an ideal distance from the chest for an appropriate signal for the MCG and sound amplifier. For example, in one embodiment two or more lightweight lasers may be used that define an intersecting crosshair at the point of ideal distance. An acoustic signal or LED-based signal to signify appropriate distance may also be used.

Accordingly, based on the Applicants' discoveries, another method pertains to a method of acquiring vital signs of a patient without contacting the patient. In a specific embodiment, the method includes placing a medical instrument having a sound sensor and/or MCG and a non-contact temperature sensor at a predetermined acquisition distance from the patient. The acquisition distance may be but is not limited to about 0.1 cm to about 500 cm from the patient. The acquisition distance may be determined by a distance signal source such as a light source configured to provide visual feedback of a predetermined distance from the patient. The method further involves obtaining one or more vital signs from the patient and optionally displaying the vital sign information on the medical instrument. In a more specific embodiment, the vital signs acquired are pulse and/or temperature or both. Sensing of temperature should not be less accurate than +/−1 degree C. Preferably, the temperature sensor provides accuracy of +/−0.5 degree C.

### ADVANTAGES OF EMBODIMENTS OF THE PRESENT INVENTION

One embodiment provides an inexpensive non-contact stethoscope and/or MCG suitable for placement in any clinical environment where vitals are taken, including ER environments or other environments where rapid assessment and triage is necessary. In one embodiment, the device is a contactless hand held portable MCG device. The inventors believe that MCG technology promises to be a helpful early screening procedure for patients who are asymptomatic for heart disease. Certain embodiments may also be used for assessing patients with symptoms of heart disease, such as chest pain, to rule out a heart attack. While multi-cell SQUID based MCGs, targeted for use by cardiologists, are already FDA cleared and commercially available, the inventors have discovered that an atomic magnetometer-based technology will enable production of an easy to use, portable device.

Furthermore, the current CPT reimbursement code 93040, defines payment for a heart rhythm acquired via a 1 to 3 lead EKG and embodiments of the invention enable a reading (via miniature LCD screen and/or wireless transmission to PC) identical to a single lead EKG without the need for lead attachment or patient contact. A contactless stethoscope may also be utilized with this contactless MCG embodiment.

In a second embodiment, a contact-less stethoscope is provided that avoids the need for an MCG element. This contactless stethoscope would address concerns about the spread of hospital acquired infections transmitted via stethoscopes.

Certain embodiments of the invention address a danger revolving around hospital acquired infections (HAIs) which claim more than 250 lives each day in the United States alone. HAIs are resulting in increased morbidity, mortality, and healthcare costs which could range from $28 billion to $30 billion each year. If all this were not sufficiently troubling for hospitals, in a significant turnabout of policy, the US Center for Medicare & Medicaid Services (CMS) issued a final rule denying hospital Medicare payments for hospital stay costs related to certain HAIs (effective Oct. 1, 2008). Moreover, most states now require mandatory public reporting of infection rates by hospital. In such states a consumer can review this information and opt for a hospital reporting the lowest rate of HAIs. Studies have shown that at least one third of all HAIs are preventable: More than 50% of all HAIs can be directly related to pathogens transmitted from patient to patient via the hands of healthcare workers. The non-contact portable vital sign detection instrument embodiments of the invention address this concern, as they function without having to touch the patient. A portable MCG, stethoscope, thermometer or any combination device_thereof is adapted according to the principles taught herein to fit this niche.

Another embodiment of the invention pertains to a general method of acquiring vital signs of a patient without contacting the patient. It is believed that the concept of acquiring vital signs has not previously been thought of before, for several reasons realized by the inventors, including but not limited to the fact that current devices that involve contact are sufficient to acquire vital signs, and thus there was not a motivation in the art to develop such device. There was no want of devices having different functionality. However, the inventors have realized that there are problems that could be addressed by providing a device which lacks the need to contact the patient, including but not limited to addressing HAIs. HAIs could be addressed significantly by providing a single device that takes the place of multiple devices for acquiring vital signs and which does not require that the patient be contacted during acquisition of such vital signs. Alleviating the need to contact the patient for this purpose decreases the amount of transmission of infectious agents.

According to certain embodiments, optical magnetometers used in conjunction with the present invention possess the following features:

Adequate Sensitivity: MCG requires sensitive magnetometers, and in order to develop an optical magnetometer-based MCG the inventors have surmised that the MCG must match the sensitivity of the current SQUID based technology. Current operational SQUID systems using liquid nitrogen have a sensitivity of 10-100 fT/√Hz. With a sensitivity of 100 fT/√Hz and a bandwidth of 100 Hz, this will produce 4-5 pt fT/√Hz peak noise, which should give us adequate sensitivity to resolve the cardiac p-wave. Atomic magnetometers have clearly shown the capacity to reach these sensitivities. For example, Romalis et al reported using a large (table sized) atomic magnetometer with a sensitivity of 0.54 fT/√Hz. More compact devices are also possible. In 2007, Schwindt, Kitching, and others described a microfabricated chip scale (<12 mm3) atomic magnetometer with sensitivity of 70 fT/√Hz, and a theoretical sensitivity of 10 fT/√Hz. Adequate sensitivity at an appropriate frequency range is required.

Adequate Frequency Response: The inventors have surmised that it is important to eliminate low frequency sources of noise. High overall sensitivity has been shown with a number of different versions of the atomic magnetometer. For example, a sensitivity of 70 fT/√Hz has been shown with chip scale versions of this magnetometer,9 and a reported sensitivity of <1 fT/√Hz by Romalis, et al. was shown for a large (table sized) unit.7,8 Sensitivity in an Mx magnetometer (similar to that used by Bison et. al.) of as low as 15 fT/√Hz was shown by Groeger et. al.15 Closer inspection, however, of the work of Romalis, et al. shows that sensitivity of their large unit is frequency dependant, with markedly increased sensor noise at lower frequencies. The reason for this loss of sensitivity at low frequencies is not discussed in the literature but the inventors have discovered that it is likely due to shot-noise related to the laser and perhaps some temperature drift. Shot-noise from a laser is a potential factor in developing adequate sensitivity across the entire frequency range in any atomic magnetometer system. We recognize that in some applications enhanced low frequency sensitivity is an important performance factor. This can be addressed by using highly stable lasers with feedback control of intensity designed for low frequency stability and by using more stable temperature control to control the temperature very stably (for example within 0.1° C. Adequate Spatial Response: The high sensitivity of optical magnetometers as noted make them attractive biosensors, but currently described atomic magnetometers have some vector specific characteristics to sensitivity. Vector specific nature of the sensors will present some technical challenges that must be accounted for. For example, while the Earth's magnetic field is a steady DC field and can be discriminated from AC biomagnetic fields on that basis, small vibrations of a vector sensitive sensor in that field result in spurious AC signals. These signals can be much larger than those to be measured. A rotation of only 1° in the Earth's field would result in a signal on the order of 600 nT assuming a perfectly vectoral sensor. The inventors have surmised that using an atomic magnetometer design that functions primarily as a scalar sensor is therefore desirable in some embodiments, including some portable embodiments. Currently described optical magnetometers do not have purely scalar detection, making them highly susceptible to vibration. A number of methods, however, are available to make sensors more globally sensitive to magnetic signals, including the following:

a.) Increasing the Number of Lasers: Currently, optical magnetometer systems use measurement lasers in configurations that lend to a directional or nodal sensitivity pattern. This directional sensitivity pattern is subject to motion artifacts as noted above. Utilization of multiple lasers enables the offsettingof nodes of lower sensitivity, and mitigate directional sensitivity, which results in a more scalar sensor than is currently described in the state of the art. A more scalar sensor is resistant to motion artifacts due to motion across ambient static magnetic fields.

b.) Creating Dynamic Spatial Response: Currently, optical magnetometer systems have static sensitivity detection profiles. A magnetometer is provided that has a dynamic directional sensitivity profile. This is achieved by a number of means, including creating a known fluctuating magnetic field with known characteristics in the range of the sensor (such as having a small rotating magnet near the sensor—a number of other means of creating a known fluctuating field will occur to those skilled in the art). Another method of creating a dynamic spatial response for a sensor would be to use a single or multiple lasers, but sweep the lasers across a known distance within the sensor to create a dynamic spatial sensitivity pattern. Creating a dynamic spatial response at an appropriate frequency (such as above the frequency of sensor vibration) can allow vibration artifacts to be mitigated. This can also be done with “lock-in” detection where the signal of interest in intentionally given a known frequency and the response signal filter is locked in to only that frequency. This effectively cancels out a large portion of the random fluctuations from vibrations.

c.) Shielding: Improving spatial response in some embodiments also includes improving spatial response to the signal of interest. In some embodiments, this will involve partially shielding the sensor to mitigate sensitivity to magnetic fields that are not in the direction of the desired target (e.g. in some embodiments the human heart). Passive shielding such as mu metal and/or ferrite shielding may be used. Active shielding using field cancellation may also be used. Creating a fluctuating magnetic field with a known period and frequency (“dynamic periodic shielding”) is another version of creating a dynamic spatial response, as is described in “b” above and may also be used to mitigate sensor spatial response.

Adequate Dynamic Range: Atomic magnetometers as noted above have many advantages in terms of cost over traditional cryogenic SQUID based sensors. However, SQUID sensors are sensitive over a wide dynamic range with excellent linearity. Dynamic range of atomic magnetometer systems can be relatively limited, dependant on the type of magnetometer used. Using an atomic magnetometer with the appropriate dynamic range will be required.

Spatial Response: The high sensitivity of optical magnetometers as noted make them attractive biosensors, but the atomic magnetometers have some vector specific characteristics to sensitivity. Vector specific nature of the sensors will present some technical challenges that will need to be overcome. For example, while the Earth's magnetic field is a steady DC field and can be discriminated from AC biomagnetic fields on that basis, small vibrations of a vector sensitive sensor in that field result in spurious AC signals. These signals can be much larger than those to be measured. A rotation of only 1° in the Earth's field would result in a signal on the order of 600 nT assuming a perfectly vectoral sensor. Using an atomic magnetometer design that functions primarily as a scalar sensor is desirable in some embodiments.

In some embodiments, an optical magnetometer is desired to generate the magnetic field information. The fundamental measurement in an atomic magnetometer is the relaxation of the alignment of magnetic atomic spins in response to an external magnetic field. There are a number of configurations described in the literature for measuring this relaxation, but in order to measure it, the atomic spins must first be aligned. This requires a circularly polarized light source, such as a laser with associated simple optics. Generally speaking, the magnetometers can be grouped into two categories: those which measure the splitting of magnetically sensitive quantum states, and magnetometers that rely on measurement of the effect of external field upon the Larmor precession of the atomic spin frequency and/or phase with an external resonant excitation. In practice, the latter configuration is much more sensitive, and that sensitivity may be helpful for our application. One embodiment of a magnetometer configuration is the Mx configuration. The Mx configuration is based on the measurement of the Larmor precession frequency. This is the frequency at which the atomic spin precess in an applied magnetic field. Mx magnetometers have a high enough sensitivity to detect brain magnetic waves, but operate successfully in the presence of large external fields and have a larger solid angle in their spatial sensitivity (closer to scalar response). In one embodiment, an Mx sensor will be used.

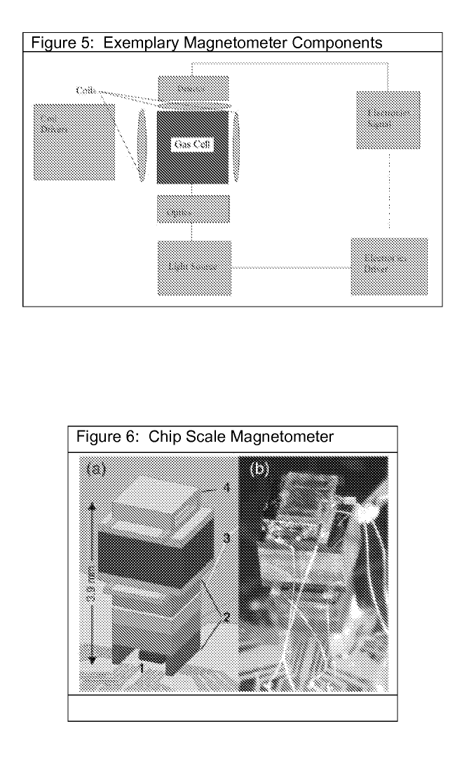

The central component (red) of one embodiment of an atomic magnetometer is a gas cell (FIG. 5). The gas cell contains isotopically enriched rubidium87 that has specific nuclear magnetic properties allowing it to be used as a magnetometer. The gas may also substitute or include other atoms with magnetically sensitive electron spins such as Cs or Na. In Mx magnetometers, the gas cell is surrounded by at least one magnetic coil creating an oscillating local magnetic bias. An external circularly polarized laser is passed through the gas cell, causing the magnetic spins of the Rb atoms to line up. The applied oscillating magnetic field in resonance with the Larmor frequency of the precession of the aligned Rb atoms. This causes the atoms to precess in phase with one another, causing the absorption of the cell, and therefore the signal at the detector to oscillate at the same frequency. The frequency is phase locked to the cell absorption. Any external magnetic field results in a phase shift of the atomic precession from the applied radio frequency (RF) bias. This phase shift is detected and processed to determine the unknown magnetic field. Other magnetometer designs are possible.

An important component of the magnetometer system is the gas cell. Gas cells used in atomic magnetometers of the present invention are simply containers with either Rb or Cs vapor and a buffer the figure is fabricated from a silicon wafer with glass windows anodically bonded to each side after deposition of a small amount of 87Rb is deposited into the cell. In addition to the 87Rb, the cell also contains a buffer gas, typically a mixture of Argon and Neon. In one example, slightly larger cells constructed of glass with about a 2 cm path are used. One example of cells that can be used include, but are not limited to, commercially available cells.

Also, used are smaller more miniaturized versions. The gas cell also requires a heating system to keep the 87Rb vapor pressure high enough to provide good absorption. The temperature must be very precisely controlled, since there is a temperature dependant offset term to the overall response of the magnetometer cell that must be calibrated. Temperature stability within 1oC post calibration is critical. This will be provided via a closed loop fluid heating system using a non-polar fluid to prevent any currents that would be a source of background magnetic fields in the vicinity of the cell. The use of a fluidic heating system also allows for measurement of the temperature of the fluid at a distance from the cell, where the temperature measurement can be performed without generation of magnetic fields in the vicinity of the cell. If the cell temperature needs to be measured directly, it can be done optically, or using multiplexing techniques. The temperature of the cell is typically held between 100oC and 140oC. (Note that this high temperature will not be an issue in the final MCG device, since only the small gas cell needs to be heated. In a commercial version, the surface of the device will be at room temperature).

The laser and optics are used to excite a 5S electron in the 87Rb atom to the 5P½ state. The absorption wavelength for this transition is 795 nm, which can be provided by a commonly available, inexpensive VCSEL. The VCSEL provides linearly polarized light which is first passed through an attenuator. The attenuator is necessary to limit the “excess' photons in the system. Some of the noise associated with the system comes from the amplitude noise from the laser, so any excess laser intensity beyond the intensity which can be absorbed by the gas cell results in excess noise without additional

signal. After the attenuator, the laser passes through a quartz quarter-wave plate to convert the linearly polarized light into circularly polarized light, as required for the transition. There is a last optical element consisting of a microlens which can columnate the beam to a width small enough to provide a high intensity beam (typically about 170 um) that is capable of providing the population trapping in the 5P½ state.

The electronics of the magnetometer may include the drive electronics for the laser. The laser is locked to the absorption of the cell by a wavelength tuning circuit. The additional drive coil is driven at an RF frequency that is in resonance with the precession frequency of the atoms that have been excited by the laser. The RF coil causes the atoms to precess in phase with one another, whereas, without such excitation, the atoms would all be out of phase. Any addition of an external magnetic field will cause the atoms to precess at slightly different phase. This out of phase condition is reflected by a modulation of the laser. The output of the photodiode, then, is detected using a phase sensitive lock-in amplifier to provide an output signal that is proportional to the unknown magnetic field. The magnetometer prototype construction is an improvement of existing technology16,17,18,19,20,21 and will primarily consist of engineering from current laboratory scale devices to provide a bench-scale, single element prototype sensor applicable to biomagnetometry.

### Environmental Noise:

While one issue is related to the sensitivity, frequency response, dynamic range, and directional response of the sensors, insufficient sensitivity related to environmental and system noise may also interfere with signal. To address this, the electronics and particularly the oscillators must be constructed using low noise techniques. Particular attention must be paid to the phase noise, as these systems are all phase sensitive and phase sensitive detection is used. If it is discovered that there is too much noise in the system, then several techniques are employed. For optical system noise, differential detection techniques are often successful at improving signal-to-noise.]

Another typical source of noise in these systems is amplitude modulation noise in the lasers. This is minimized by keeping the laser power to the minimum necessary to invert the population of the gas cell. If necessary, some amplitude noise is corrected for using reference techniques.

A third source of potential noise is the Johnson noise from the shielding itself. This is solved by using different shielding materials or by keeping the shielding far from the sensors.

At very low frequencies, other potential sources of noise include drift of temperature control and laser long term stability. These are considered early in the design phase, but are also checked in the experimental sensor.

### REFERENCES

¹Merlin M A, Wong M L, Pryor P W, Rynn K, Marques-Baptista A, Perritt R, Stanescu C G, Fallon T. Prevalence of methicillin-resistant Staphylococcus aureus on the stethoscopes of emergency medical services providers. Prehosp Emerg Care 2009;13:71-74.

²Madar R, Novakova E, Baska T, The role of non-critical health-care tools in the transmission of nosocomial infections. Bratisl Lek Listy, 2005; 106(11):248-50.

³Whittington A M, Whitlow G, Hewson D, Thomas C, Brett S J, Bacterial Contamination of Stehtoscopes on the intensive care unit, Anaesthesia. 2009 June;64(6):620-4.

⁴I Youngster, M Berkovitch, E Heyman, Z Laxarovitch, M Godlman, The stethoscope as a vector of infectious diseases in the paediatric division, Acta Pdiatrica/Acta Pdiatrica 2008 97, pp. 1253-1255

⁵Williams, C. Davis D L, Methicillin-resistant Staphylococcus aureus fomite survival. Clin Lab Sci. 2009: 22(1): 34-8.

⁶Allevne S A, Hussain A M, Clokie M, Jenkins DR Stethoscopes: potential vectors of Clostridium difficile. J Hosp Infect. 2009 October;73(2):187-9. Epub 2009 Aug 28.

⁷Levin M J, Leventhal S, Masters H A, Factors influencing quantitative isolationof varicell-zoster virus. J Clin Microbiol. 1984 June;19(6):880-3.

⁸Gastmeier P, Groneberg K., Weist K, A Cluster of nonsocomial Klebsiella pneumonia bloodstream infections in a neonatal intensive care department: Identification of Transmission and intervention, Am J. Infect Control, 2003, November 31(7): 424-30

⁸Wood M W, Lund R C, Stevenson K B, Bacterial contamination of stethoscopes with antimicrobial diaphragm covers, Am. J. Infect Control 2007 May: 35(4) 263-6.

⁹Lecat P, Cropp E, McCord G, Haller N A, Ethanol-based cleanser versus isopropyl alcohol to decontaminate stethoscopes. Am J. Infect Control 2009 April: 37(3) 241-3. Epub 2009 Jan. 30.

¹²Schwindt P D D, Knappe S, Shah V, Hollberg L, Liew L A, Moreland J, Kitching J. Chip-scale atomic magnetometer. Applied Physics Letters 2004; 85 (26-27), 6409-6411.

¹³Schwindt P D D, Lindseth B, Knappe S, Shah V, Liew L A, Kitching J. Chip-scale atomic magnetometer with improved sensitivity by use of the Mx technique. Applied Physics Letters 2007; 90; 7501-7504.

¹⁴Shah V, Knappe S, Schwindt P D D, Kitching J. Subpicotesla atomic magnetometry with a microfabricated vapour cell. Nature Photonics 2007; 1: 649-652.

¹⁵Groeger S, Bison G, Schenker J L, Wynands R, Weis A. A high-sensitivity laser-pumped Mₓ magnetometer. The European physical journal. D, Atomic, molecular and optical physics 2006; 38 (2): 239-247.

¹⁶Brannon A, Shah V, Popović1 Z. Self-Injection Locking of a Microwave Oscillator by Use of Four-Wave Mixing in an Atomic Vapor. IEEE 2007: 275-278.

¹⁷Gerginov V, Knappe S, Shah V, Kitching J, Hollberg L. Reduction of optical field noise by differential detection in atomic clocks based on coherent population trapping. Proc. SPIE 2006; 6604: 66040 J 1-5.

¹⁸Gerginov V, shah V, Knappe S, Hollberg L, Kithcing J. Atomic-based stabilization for laser-pumped atomic clocks. Optics Letters 2006; 31(12): 1851-3.

¹⁹Knappe S, Shah V, Brannon A, Gerginov V, Robinson HG, PopovićZ, Hollberg L, Kitching J. Advances in Chip-Scale Atomic Frequency References at NIST. Proc. of SPIE Vol. 6673, 667307, (2007)

²⁰Schwindt P D D, Lindseth B, Knappe S, Shah V, Kitching J. Chip-scale atomic magnetometer with improved sensitivity by use of the Mx technique. Applied Physics Letters 2007; 90, 081102

²¹Shah V, Gerginov V, Schwindt P D D, Knappe S, Hollberg L, Kitching J. Continuous light-shift correction in modulated coherent population trapping clocks. Applied Physics Letters 2006; 89, 151124.

What is claimed is:

1. A portable, handheld device for assessing vital signs without needing to contact a subject or any object in contact with the subject, the device comprising singly a magnetometer, singly an acoustic transducer or either or both in combination with a non-contact body temperature measurement device, and at least one battery component.

2. The device of claim 1, wherein the device comprises a magnetometer and acoustic transducer, wherein said at least one battery component is connected to said magnetometer.

3. A non-contact stethoscope comprising an acoustic transducer for detection of acoustic signals without contacting the subject or any object in contact with the subject.

4. The device of claim 3 where the acoustic transducer is coupled with concentrator for focusing acoustic signals.

5. The device of claim 4, wherein said concentrator is dimensioned for focusing acoustic signals toward said acoustic transducer based on size of an acoustic signal generating portion of said subject and based on a predetermined distance range for obtaining acoustic signals from said subject.

6. The device of claim 5, wherein said concentrator is sized to focus acoustic signals from said acoustic signal generating portion of said subject of a size 3 feet or less in its broadest dimension.

7. The device of claim 5, wherein said concentrator is sized to focus acoustic signals from said acoustic signal generating portion of said subject at a distance of from about 0.1 inches to 10 feet away, or any specific inch increment therebetween, from said device.

8. Device of claim 4 where the concentrator is a substantially parabolic reflector

9. Device of claim 4 where the concentrator is a waveguide.

10. Device of claim 9 where the waveguide has some tapered component.

11. Device of claim 3 where the acoustic transducer is a microphone element.

12. Device of claim 11 where the microphone element comprises one of the list of
a. Electret microphone
b. Condenser microphone
c. Optical microphone

13. Device of claim 3 with the inclusion of an acoustic-blocking material that blocks some extraneous acoustic energy.

14. Device of claim 3 where the signal is processed to accentuate sounds from a desired source.

15. Device of claim 14 where the source includes one or more of
a. Heart sounds
b. Lung sounds
c. Gut sounds
d. Throat sounds
e. Vascular sounds

16. A non-contact stethoscope comprising an acoustic concentrator coupled to a tube for delivery of the sound waves to the auscultator without the concentrator contacting the subject or any object in contact with the subject.

17. Device of claim 16 with the inclusion of an acoustic-blocking material that blocks some extraneous acoustic energy.

18. The device of claim 1 where the signals obtained are transmitted wirelessly to a receiving device.

19. The device of claim 18 where the signal is transmitted via radio waves

20. The device of claim 18 where the signal is transmitted via optical energy

21. The device of claim 1 including a magnetic field detector

22. The device of claim 1 including a non-contact stethoscope.

23. The device of claim 1 including a non-contact body temperature measurement device

24. The device of claim 21 where the magnetic field detector is one of
a. Optical magnetometer
b. Solid State magnetometer

25. The device of claim 21 including magnetic shielding

26. The device of claim 21 where the magnetometer is a scalar magnetometer

27. The device of claim 21 where the magnetometer is an optical magnetometer using a solid state light source

28. A method for optimizing the operational distance of the non-contact measurement system from the subject including two or more non-parallel visible light beams intersecting or otherwise coming in to a predetermined pattern when projected from a distance near the optimum distance from the subject.

29. The device of claim 1 including a device for optimizing the operational distance from the subject.

30. The device of claim 29 where the device for optimizing the distance is one or more non-parallel visible light beams designed to cross or otherwise come in to an alignment pattern when projected to the subject near the optimal operational distance.

31. A method of acquiring vital signs of a patient without contacting the patient, said method comprising placing a medical instrument having an acoustic transducer at a predetermined acquisition distance from the patient.

32. A portable, handheld device for assessing vital signs without needing to contact a subject or any object in contact with the subject, the device equipped to acquire signals comprising singly magnetic signals, singly acoustic signals, or either or both in combination with temperature information from said subject or any object in contact with the subject.
